(19) United States
(12) Patent Application Publication
(10) Pub. No.: US 2025/0352679 A1
(43) Pub. Date: Nov. 20, 2025

(54) IN-SITU STERILIZATION IN VAPOR PHASE DEPOSITION

(71) Applicant: Applied Materials, Inc., Santa Clara, CA (US)

(72) Inventors: Shrikant Swaminathan, Santa Clara, CA (US); Sukti Chatterjee, San Jose, CA (US); Lance A. Scudder, Sunnyvale, CA (US); Ravindra Patil, Santa Clara, CA (US)

(21) Appl. No.: 19/207,224

(22) Filed: May 13, 2025

Related U.S. Application Data
(60) Provisional application No. 63/649,163, filed on May 17, 2024.

(51) Int. Cl. A61L 2/00; A61L 101/02; C23C 16/02; C23C 16/44; C23C 16/455

(52) U.S. Cl. CPC A61L 2/0094; C23C 16/02; C23C 16/4408; C23C 16/45555; A61L 2101/02; A61L 2202/21

(57) ABSTRACT

The disclosure is directed at methods for reducing the bioburden of both particles comprising an active pharmaceutical ingredient (API) and the equipment used to process such particles.

### CROSS-REFERENCE TO RELATED APPLICATIONS

This application claims priority to U.S. Provisional Application No. 63/649,163, filed on May 17, 2024, the disclosures of which are incorporated by reference.

### TECHNICAL FIELD

The present disclosure relates to methods for reducing the bioburden of both particles comprising an active pharmaceutical ingredient (API) and the equipment used to process such particles.

### BACKGROUND

Lowering the bioburden of active pharmaceutical ingredients is of importance in the pharmaceutical industry, and is particularly important in the case of an API that is used in an injectable or intravenous dosage form. In addition, is it important that equipment use to process API have a low bioburden.

### SUMMARY

In one aspect, the disclosure is related to a method of applying a coating to particles comprising an active pharmaceutical ingredient (API), the method comprising:
(a) loading the particles into a chamber;
(b) applying a vaporous or gaseous metal or metalloid precursor to the particles in the reactor by pulsing the vaporous or gaseous aluminum precursor into the reactor;
(c) performing one or more pump-purge cycles using an inert gas;
(d) applying a vaporous or gaseous oxidant to the particles in the reactor by pulsing the oxidant into the reactor;
(e) performing one or more pump-purge cycles using an inert gas;
(f) repeating steps (e)-(h) at least once to provide a metal oxide or metalloid oxide coating layer on the particles;
and a sterilization step before step (a), between step (a) and step (b), and/or after step (f).

In some embodiments, the sterilization step comprises isolating the chamber from the ambient environment.

In some embodiments, the method comprises a sterilization step between step (a) and step (b).

In some embodiments, the sterilization step comprises: injecting ozone into the chamber to reach a predetermined ozone concentration and holding for a predetermined period of time.

In some embodiments, the sterilization step further comprises removing the ozone after the expiry of the predetermined period of time.

In some embodiments, the removing further includes injecting an inert gas into the chamber.

In some embodiments, the inert gas is nitrogen, argon, or helium.

In some embodiments, the predetermined ozone concentration is 9-19 ppm, and the method further comprises injecting water vapor into the chamber to reach a predetermined steam pressure and holding for a predetermined period of time.

In some embodiments, the predetermined ozone concentration is 30-120 ppm.

In some embodiments, the predetermined period of time is less than 1 hour.

In some embodiments, the predetermined period of time is 3-5 hours.

In some embodiments, the predetermined period of time is 5-10 minutes, and the method further comprises injecting water vapor into the chamber to reach a predetermined steam pressure and holding for a predetermined period of time.

In some embodiments, the sterilization step comprises: injecting H2O2 into the chamber to reach a predetermined H2O2 concentration and holding for a predetermined period of time.

In some embodiments, the sterilization step further comprises removing the ozone after the expiry of the predetermined period of time.

In some embodiments, the removing further includes injecting an inert gas into the chamber.

In some embodiments, the inert gas is nitrogen, argon, or helium.

In some embodiments, the predetermined H2O2 concentration is 100-500 ppm.

In some embodiments, the predetermined H2O2 concentration is 400-6000 ppm, and the method further comprises injecting water vapor into the chamber to reach a predetermined steam pressure and holding for a predetermined period of time.

In some embodiments, the predetermined period of time is less than 1 hour.

In some embodiments, the predetermined period of time is 30-80 minutes.

In some embodiments, the predetermined period of time is in the range of from 10 minutes to 50 minutes, and the method further comprises injecting water vapor into the chamber to reach a predetermined steam pressure and holding for a predetermined period of time.

In some embodiments, the sterilization step comprises increasing the temperature of the chamber to reach a target temperature and maintaining the target temperature for a predetermined period of time.

In some embodiments, the target temperature is 350° C.

In some embodiments, the predetermined period of time is in the range of from 15 minutes to 5 hours.

In some embodiments, the predetermined period of time is 15-60 minutes.

In some embodiments, the sterilization step further comprises decreasing the temperature of the chamber after the expiry of the predetermined period of time.

In some embodiments, the sterilization step comprises: injecting water vapor into the chamber to reach a predetermined steam pressure and holding for a predetermined period of time.

In some embodiments, the sterilization step further comprises removing the water vapor after the expiry of the predetermined period of time.

In some embodiments, the predetermined steam pressure is 700 Torr.

In some embodiments, the predetermined period of time is in the range of from 30 minutes to 5 hours.

In some embodiments, the predetermined period of time is 30-90 minutes.

In some embodiments, the bio-burden of the particles is reduced by more than 80%.

In some embodiments, the bio-burden of the particles is reduced by more than 90%.

In some embodiments, the particles are agitated during at least a portion of the process.

In some embodiments, the metal oxide or metalloid oxide layer has a thickness in range of 0.1 nm to 100 nm.

In some embodiments, the particles comprise one or more pharmaceutically acceptable excipients.

In some embodiments, the particles have a median particle size, on a volume average basis between 1 μm and 1000 μm.

In some embodiments, the pharmaceutical composition is removed from the chamber and admixed with a pharmaceutically acceptable diluent or carrier.

In some embodiments, the particles consist essentially of the API or excipient.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting.

Other features and advantages of the invention will be apparent from the following detailed description and figures, and from the claims.

### BRIEF DESCRIPTION OF THE DRAWINGS

FIG. 1 is a schematic illustration of a rotary reactor useful for applying coatings to drug containing particles using a vapor phase deposition process such as atomic layer coating.

FIG. 2 is a schematic illustration of an exemplary process for sterilizing particles comprising an API.

FIG. 3 is a schematic illustration of an exemplary process for sterilizing particles comprising an API.

FIG. 4 is a schematic illustration of an exemplary reaction chamber structure.

FIG. 5A is an exemplary image of lactose powder inside a reaction chamber before sterilization using a wet ozone process.

FIG. 5B is an exemplary image of lactose powder inside a reaction chamber after sterilization using a wet ozone process.

FIG. 6 shows exemplary cycles of the wet ozone sterilization process and the dry ozone sterilization process.

FIG. 7A-7B show bioburden testing data from the dry ozone process (FIG. 7A) and the wet ozone process (FIG. 7B).

### DETAILED DESCRIPTION

The present disclosure provides methods of reducing the bioburden of particles comprising an API and certain equipment used to process particles comprising an API that are subsequently treated using a vapor phase deposition process, for example, an atomic layer coating process or molecular layer deposition process, to apply a thin coating of, for example, a metal oxide. Importantly, the entire process can occur in a reaction chamber so that once the particles are treated to reduce their bioburden, the particles remain within the reaction chamber through the subsequent coating process. The process entails exposing the API containing particles to ozone or H₂O₂ vapor in a reaction chamber. In either case, water vapor can also be present. Thus, there is a dry ozone process, a wet ozone process, a wet H₂O₂ process and a dry H₂O₂ process. After the API particles have been treated by one of these process within the reaction chamber, the ozone or H₂O₂ vapor (and any water vapor present) is removed by purging the reaction chamber with an inert gas (e.g., nitrogen) the vapor phase deposition process is then carried out without removing the particles from the reaction chamber.

The term “pulse” or “pulsing” may be understood to comprise feeding reactant (or purge gas or another gas) into the reaction chamber for a predetermined amount of time.

### Active Pharmaceutical Ingredient

Active pharmaceutical ingredients (API) that can be treated using the methods described herein include: small molecule drugs, viruses, polypeptides, polynucleotides, a composition comprising polypeptide and lipid, and a composition comprising polynucleotide and lipid. The API can be selected from the group consisting of an analgesic, an anesthetic, an anti-inflammatory agent, an anthelmintic, an anti-arrhythmic agent, an antiasthma agent, an antibiotic, an anticancer agent, an anticoagulant, an antidepressant, an antidiabetic agent, an antiepileptic, an antihistamine, an antitussive, an antihypertensive agent, an antimuscarinic agent, an antimycobacterial agent, an antineoplastic agent, an antioxidant agent, an antipyretic, an immunosuppressant, an immunostimulant, an antithyroid agent, an antiviral agent, an anxiolytic sedative, a hypnotic, a neuroleptic, an astringent, a bacteriostatic agent, a beta-adrenoceptor blocking agent, a blood product, a blood substitute, a bronchodilator, a buffering agent, a cardiac inotropic agent, a chemotherapeutic, a contrast media, a corticosteroid, a cough suppressant, an expectorant, a mucolytic, a diuretic, a dopaminergic, an antiparkinsonian agent, a free radical scavenging agent, a growth factor, a haemostatic, an immunological agent, a lipid regulating agent, a muscle relaxant, a protein, a peptide, a polypeptide, a parasympathomimetic, a parathyroid calcitonin, a biphosphonate, a prostaglandin, a radio-pharmaceutical, a hormone, a sex hormone, an anti-allergic agent, an appetite stimulant, an weight loss agent, a steroid, a sympathomimetic, a thyroid agent, a vaccine, a vasodilator and a xanthine. Preferred API are organic API.

Exemplary small molecule drugs include, but are not limited to, acetaminophen, clarithromycin, azithromycin, ibuprofen, fluticasone propionate, salmeterol, pazopanib HCl, palbociclib, and amoxicillin potassium clavulanate. Exemplary types of polypeptide drugs include, but are not limited to, proteins (e.g., antibodies), peptide fragments (e.g., antibody fragments), alemtuzumab, bevacizumab, cetuximab, gemtuzumab ozogamicin, ipilimumab, ofatumumab, panitumumab, pembrolizumab, ranibizumab, rituximab, or trastuzumab. Exemplary types of polynucleotide drugs include, but are not limited to, one or more of DNA, RNA including messenger mRNA (mRNA), hybrids thereof, RNAi-inducing agents, RNAi agents, siRNAs, shRNAs, miRNAs, antisense RNAs, ribozymes, catalytic DNA, triple helix formation inducing RNAs, aptamers, and vectors. Exemplary types of lipids include, but are not limited to fats, waxes, sterol-containing metabolites, vitamins, fatty acids, glycerolipids, glycerophospholipids, sphingolipids, saccharolipids, and polyketides, and prenol lipids.

In the present disclosure, the drug loaded into the reactor may be in in the form of particles. Exemplary methods of preparing drugs in particulate form include, but are not limited to, processes utilizing dry or wet milling, lyophilization, freeze-drying, precipitation, and dry compacting.

### Vapor Phase Deposition

The coatings can be applied by vapor phase deposition (also called atomic layer coating) using a precursor molecule and an oxidant (e.g., ozone or water vapor). Vapor phase deposition (atomic layer coating) of metal oxides and metalloid oxides is sometimes referred to as atomic layer deposition (ALD). However, depending on a number of factors, including the temperature of the reaction, each cycle of the deposition reaction does not necessarily deposit one atomic layer and the deposition per cycle is not necessarily constant. Exemplary metal oxide and metalloid oxide coating that can be deposited by atomic layer coating include, but are not limited to, aluminum oxide, titanium dioxide, silicon oxide, and zinc oxide. Also included are iron oxide, gallium oxide, magnesium oxide, niobium oxide, hafnium oxide, tantalum oxide, lanthanum oxide, and zirconium dioxide. Exemplary oxidants include, but are not limited to, water, ozone, and inorganic peroxide.

### Chemical Vapor Deposition (CVD)

Chemical vapor deposition is a thin-film deposition technique by which an element or chemical compound is deposited on a surface by chemical reaction in the gas phase or on a surface. It is distinct from atomic layer deposition in that the deposition is not self-limited, i.e., the film will continue to grow as long as chemistry is supplied. It is distinct from physical vapor deposition in that a chemical reaction results in a deposited film that is chemically different from the precursor species.

### Reactor System

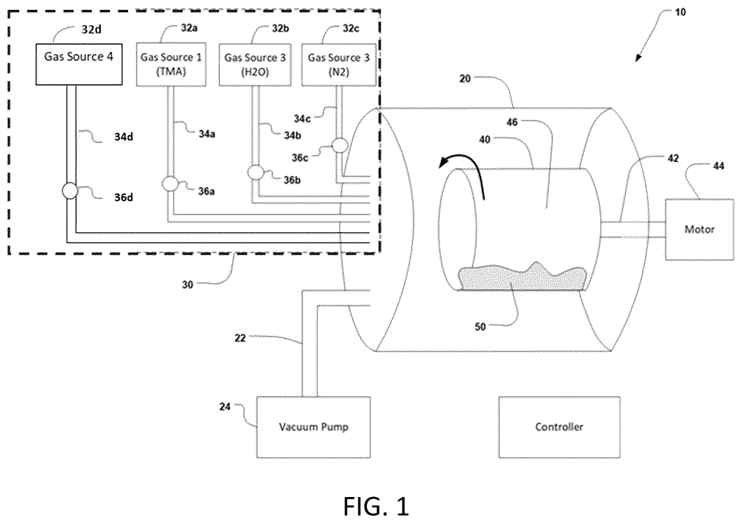

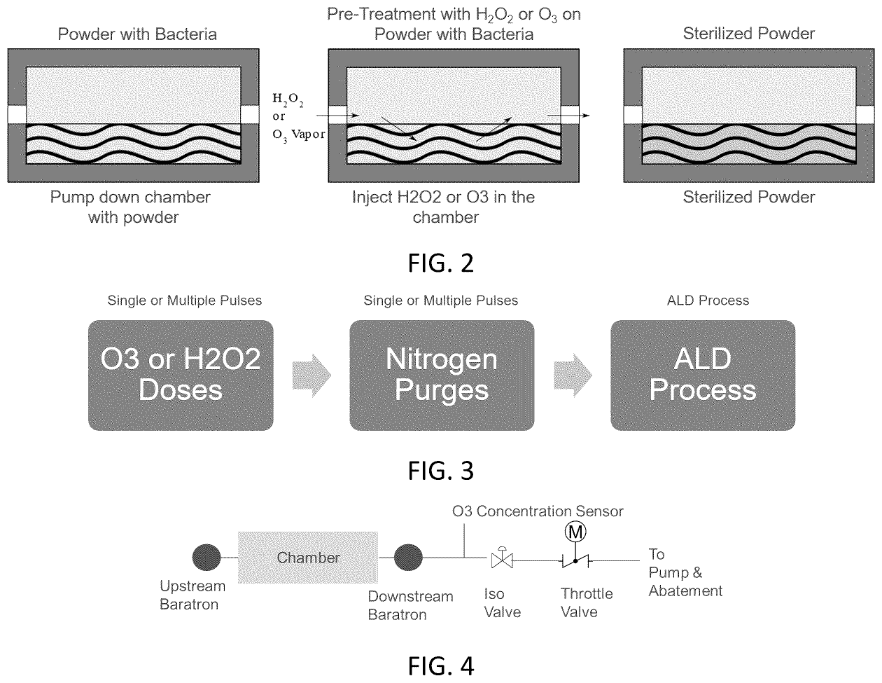

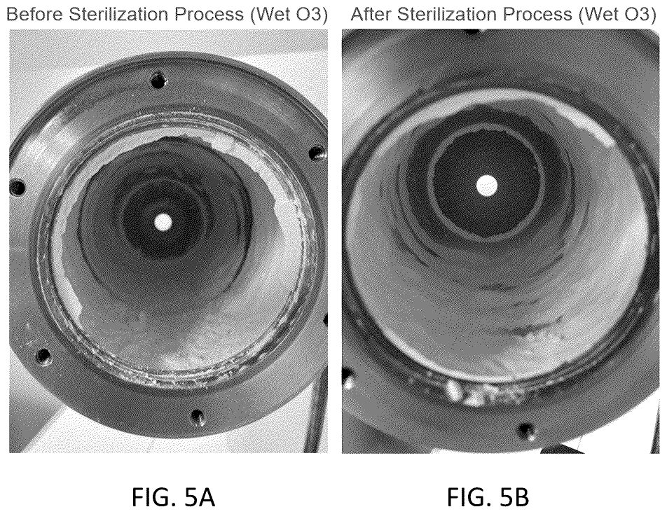

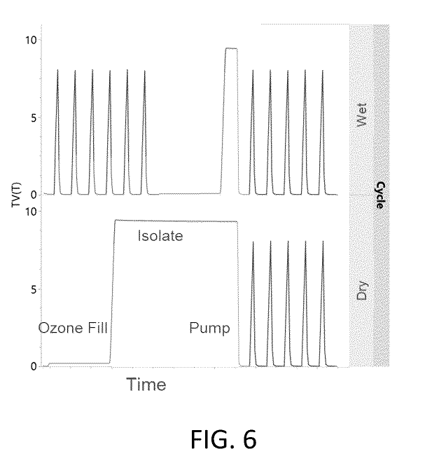

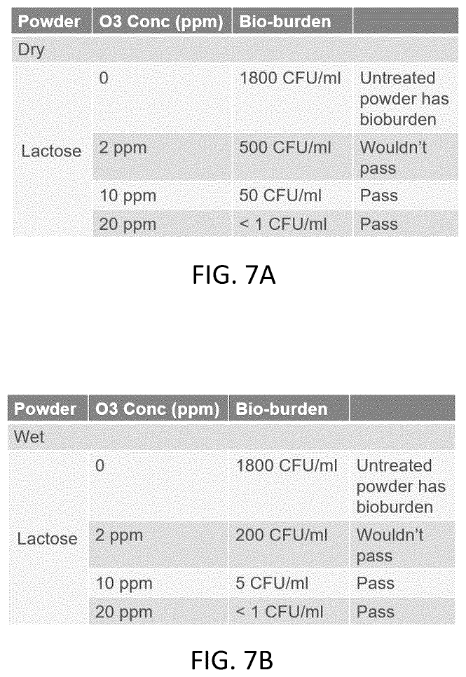

The term “reactor system” in its broadest sense includes all systems that could be used to perform vapor phase deposition, or mixed vapor phase deposition/CVD or CVD. An exemplary reactor system is illustrated in FIG. 1 and further described below.

FIG. 1 illustrates a reactor system 10 for performing coating of particles, e.g., thermally sensitive particles, with thin-film coatings. The reactor system 10 can perform the coating using vapor phase deposition and/or CVD coating conditions. The relative contribution of vapor phase deposition and CVD processes to the thin-film coating can be controlled by appropriate selection of process conditions. In particular, the reactor system 10 permits a primarily vapor phase deposition process, e.g., an almost entirely vapor phase deposition process, to be performed at low processing temperature, e.g., below 50° C., e.g., at or below 35° C. For example, the reactor system 10 can form thin-film metal oxides on the particles primarily by ALD at temperatures of 22-35° C., e.g., 25-35° C., 25-30° C., or 30-35° C. In general, the particles can remain or be maintained at such temperatures. This can be achieved by having the reactant gases and/or the interior surfaces of the reactor chamber (e.g., the chamber 20 and drum 40 discussed below) remain or be maintained at such temperatures.

Performing vapor phase deposition reaction at low temperature conditions permits coatings to be formed on the particles without degradation of the biological components, e.g., the vaccine or bio-pharma ingredients. For example, a biological component in amorphous form can be coated without breaking down the biological component or converting the biological component to a crystalline form.

The reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure, and permits removal of reaction byproducts and unreacted process gases.

In operation, the reactor 10 performs the vapor phase deposition thin-film coating process by introducing gaseous precursors of the coating into the chamber 20. The gaseous precursors are spiked alternatively into the reactor. This permits the vapor phase deposition process to be a solvent-free process. The half-reactions of the vapor phase deposition process can be self-limiting, which can provide Angstrom level control of deposition. In addition, the vapor phase deposition reaction can be performed at low temperature conditions, such as below 50° C., e.g., below 35° C.

The chamber 20 is also coupled to a chemical delivery system 30. The chemical delivery system 20 includes gas sources 32a, 32b, 32c, 32d coupled by respective delivery tubes 34a, 34b, 34c, 34d and controllable valves 36a, 36b, 36c, 36d to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 1 illustrates separate gas lines extending in parallel to the chamber for each gas source, two or more of the gas lines could be joined, e.g., by one or more three-way valves, before the combined line reaches the chamber 20. In addition, although FIG. 1 illustrates three gas sources, the use of four gas sources could enable the in-situ formation of laminate structures having alternating layers of two different metal oxides.

Two of the gas sources provide two chemically different gaseous reactants for the coating process to the chamber 20. Suitable reactants include any of or a combination of the following: monomer vapor, metal-organics, metal halides, oxidants, such as ozone or water vapor, and polymer or nanoparticle aerosol (dry or wet). For example, the first gas source 32a can provide gaseous trimethylaluminum (TMA) or titanium tetrachloride (TiCl₄), whereas the second gas source 32b can provide water vapor.

One of the gas sources can provide a purge gas. In particular, the third gas source 32c can provide a gas that is chemically inert to the reactants, the coating, and the particles being processed. For example, the purge gas can be N2, or a noble gas, such as argon.

One of the gas sources can provide a sterilization gas. In particular, the fourth gas source 32d can provide a gas that can sterilize the reactor chamber. For example, the sterilization gas can be ozone or H₂O₂.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 10 μm.

In operation, one of the gasses flows into chamber 20 from the chemical delivery system 30 as the drum 40 rotates. A combination of pores (1-100 um), holes (0.1-10 mm), or large openings in the coating drum serve to confine the particles in the coating drum while allowing rapid delivery of precursor chemistry and pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to keep them separate, ensuring a large surface area of the particles remains exposed. This permits fast, uniform interaction of the particle surface with the process gas.

In some implementations, one or more temperature control components are integrated into the drum 40 to permit control of the temperature of the drum 40. For example, resistive heater, a thermoelectric cooler, or other component can in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source (32a, 32b, 32c, 32d), the rotation rate of the motor 44, etc. The controller 60 can receive the recipe as computer-readable data (e.g., that is stored on a non-transitory computer readable medium).

The controller 60 and other computing devices part of systems described herein can be implemented in digital electronic circuitry, or in computer software, firmware, or hardware. For example, the controller can include a processor to execute a computer program as stored in a computer program product, e.g., in a non-transitory machine readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general purpose programmable computer. In some implementations, the controller can be implemented using special purpose logic circuitry, e.g., an FPGA (field programmable gate array) or an ASIC (application specific integrated circuit).

### Operation

Initially, particles are loaded into the drum 40 in the reactor system 10. The particles can have a solid core comprising an API. Once any access ports are sealed, the controller 60 operates the reactor system 10 according to the recipe in order to sterilize the particles and to form the thin-film layers on the particles.

In some embodiments, a sterilization step occurs before the particles are loaded into the chamber 20. In some embodiments, a sterilization step occurs after the particles are loaded into the chamber 20. In some embodiments, a sterilization step occurs after the coating process is finished. In some embodiments, aseptic processing can be performed before the particles are loaded into the chamber 20. In some embodiments, a sterilization step occurs before the particles are loaded into the chamber 20, and after the coating process is finished.

In some embodiments, during the sterilization step, gaseous ozone or H₂O₂ is introduced into the chamber. In some cases water vapor is also introduced into the chamber. In some embodiments, during the sterilization step, the temperature of the chamber is increased. In some embodiments, during the sterilization step, water vapor is introduced into the chamber. In some embodiments, the chamber is isolated from the ambient environment for a certain period of time. After the sterilization is finished, any residual ozone or H₂O₂, and water vapor, if present, along with any byproduct, is removed from the chamber. The chamber is then purged with an inert gas, using for example, a series of pump purge cycles.

For the coating process, the two reactant gases are alternately supplied to the chamber 20, with each step of supplying a reactant gas followed by a purge cycle in which the inert gas is supplied to the chamber 20 to force out the reactant gas and by-products used in the prior step. Moreover, one or more of the gases (e.g., the reactant gases and/or the inert gas) can be supplied in pulses in which the chamber 20 is filled with the gas to a specified pressure, a delay time is permitted to pass, and the chamber is evacuated by the vacuum pump 24 before the next pulse commences.

In particular, the controller 60 can operate the reactor system 10 as follows.

In a first reactant half-cycle, while the motor 44 rotates the drum 40 to agitate the particles 50:
i) The gas distribution system 30 is operated to flow the first reactant gas, e.g., TMA, from the source 32a into the chamber 20 until a first specified pressure is achieved. The specified pressure can be 0.1 Torr to half of the saturation pressure of the reactant gas.
ii) Flow of the first reactant is halted, and a specified delay time is permitted to pass, e.g., as measured by a timer in the controller. This permits the first reactant to flow through the particle bed in the drum 40 and react with the surface of the particles 50 inside the drum 40.
   iii) The vacuum pump 50 evacuates the chamber 20, e.g., down to pressures below 1 Torr, e.g., to 1 to 100 mTorr, e.g., 50 mTorr.

These steps (i)-(iii) can be repeated a number of times set by the recipe, e.g., two to ten times, e.g., six times.

Next, in a first purge cycle, while the motor 44 rotates the drum to agitate the particles 50:
iv) The gas distribution system 30 is operated to flow the inert gas, e.g., N2, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the reactant gas and any vaporous by-products.
vi) The vacuum pump 50 evacuates the chamber 20, e.g., down to pressures below 1 Torr, e.g., to 1 to 500 mTorr, e.g., 50 mTorr.

These steps (iv)-(vi) can be repeated a number of times set by the recipe, e.g., six to twenty times, e.g., sixteen times.

In a second reactant half-cycle, while the motor 44 rotates the drum 40 to agitate the particles 50:
vii) The gas distribution system 30 is operated to flow the second reactant gas, e.g., H2O, from the source 32a into the chamber 20 until a third specified pressure is achieved. The third pressure can be 0.1 Torr to half of the saturation pressure of the reactant gas.
viii) Flow of the second reactant is halted, and a specified delay time is permitted to pass, e.g., as measured by the timer in the controller. This permits the second reactant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

These steps (vii)-(ix) can be repeated a number of times set by the recipe, e.g., two to ten times, e.g., six times.

Next, a second purge cycle is performed. This second purge cycle can be identical to the first purge cycle, or can have a different number of repetitions of the steps (iv)-(vi) and/or different delay time and/or different pressure.

The cycle of the first reactant half-cycle, first purge cycle, second reactant half cycle and second purge cycle can be repeated a number of times set by the recipe, e.g., one to ten times.

As noted above, the coating process can be performed at low processing temperature, e.g., below 50° C., e.g., at or below 35° C. In particular, the particles can remain or be maintained at such temperatures during all of steps (i)-(ix) noted above. In general, the temperature of the interior of the reactor chamber does not exceed 35° C. during of steps (i)-(ix). This can be achieved by having the first reactant gas, second reactant gas and inert gas be injected into the chamber at such temperatures during the respective cycles. In addition, physical components of the chamber of the chamber can remain or be maintained at such temperatures, e.g., using a cooling system, e.g., a thermoelectric cooler, if necessary.

### Process Using Ozone

The process can include:
1. introduction of ozone into the reactor chamber (with or without the addition of water vapor) combined with suitable humidity control and turbulent airflow;
2. measurement and control of effective exposure to ozone optimum for sterilization of the particles and the various surfaces of the reactor; and
3. removal of ozone (and water vapor, if present) and any gaseous by-products from the reactor chamber before the start of the coating process.

As an example, the method according to the disclosure can include the following steps:
a) connecting an ozone generator to the reactor chamber;
b) elevating and maintaining the ozone concentration in the reactor chamber at a level sufficient to act as a disinfectant taking into account the humidity, size, temperature and airflow of the reactor chamber;
c) removing the ozone (and water vapor, if present) and any gaseous by-products from the reactor chamber.

For example, API particles are loaded to the chamber of the reactor. Optionally, the pressure in the chamber can be reduced, for example to below 0.01 torr. Next, ozone (with or without water vapor) is introduced into the chamber at a certain concentration (e.g., 1-10 Torr). In a wet ozone process, water vapor is also introduced (e.g., at an ozone/water ratio of 1:10), and ozone can be introduced at about 9-19 ppm. In a dry ozone process, water vapor in not introduced, and ozone can be introduced at about 30-120 ppm. The ozone or ozone and water vapor is held in the chamber (e.g., isolated from the environment) for a period of time (e.g., 1-10 hours). The particles are preferably agitated during this time. For example, the chamber can be treated with wet ozone process for a shorter time (e.g., 5 minutes), or the chamber can be treated with dry ozone process for a longer time (e.g., 3-5 hours). Finally, the ozone (and water vapor, if present) and any gaseous by-products are removed from the chamber.

Ozone can be injected in a single pulse or multiple pulses. Further, ozone can be removed from the chamber by using a single pulse or multiple pulses of an inert gas (nitrogen purges). For the dry ozone process, single-pulse injection of ozone can be used and requires less time than multi-pulse injection. For the wet ozone process, multi-pulse injection is preferable because it can minimize water condensation and particle agglomeration.

Finally, the coating process can begin after the sterilization process.

Process Using H₂O₂

The process can include:
1. introduction of H₂O₂ into the reactor chamber (with or without the addition of water vapor) combined with suitable humidity control and turbulent airflow;
2. measurement and control of effective exposure to ozone optimum for sterilization of the particles and the various surfaces of the reactor; and
3. removal of H₂O₂ (and water vapor, if present) and any gaseous by-products from the reactor chamber before the start of the coating process.

As an example, the method according to the disclosure can include the following steps:
a) connecting an H₂O₂ source to the reactor chamber;
b) elevating and maintaining the H₂O₂ vapor concentration in the reactor chamber at a level sufficient to act as a disinfectant taking into account the humidity, size, temperature and airflow of the reactor chamber;
c) removing the H₂O₂ and any gaseous by-products from the reactor chamber.

For example, API particles are loaded to the chamber of the reactor. Optionally, the pressure in the chamber can be reduced, for example to below 0.01 torr. Next, H₂O₂ vapor (with or without water vapor) is introduced into the chamber at a certain concentration (e.g., below 2 Torr). In a wet H₂O₂ process, water vapor is also introduced, and H₂O₂ can be introduced at about 400-6000 ppm. In a dry H₂O₂ process, water vapor in not introduced, and H₂O₂ can be introduced at about 100-500 ppm. The H₂O₂ or H₂O₂ and water vapor is held in the chamber (e.g., isolated from the environment) for a period of time (e.g., 1-10 hours). The particles are preferably agitated during this time. For example, the chamber can be treated with wet H₂O₂ process for a shorter time (e.g., 10-50 minutes), or the chamber can be treated with dry H₂O₂ process for a longer time (e.g., 30-80 minutes). Finally, the H₂O₂ (and water vapor, if present) and any gaseous by-products are removed from the chamber.

H₂O₂ vapor can be injected in a single pulse or multiple pulses. Further, H₂O₂ can be removed from the chamber by using a single pulse or multiple pulses of an inert gas (nitrogen purges). For the dry H₂O₂ process, single-pulse injection of H₂O₂ can be used and requires less time than multi-pulse injection. For the wet ozone process, multi-pulse injection is preferable because it can minimize water condensation and particle agglomeration.

Finally, the coating process can begin after the sterilization process.

### Process Using Dry Heat

The process can include:
1. increasing the temperature of the reactor chamber to reach a target temperature (e.g., 350° C.) (it may take up to one hour to reach the desired temperature);
2. maintaining the chamber at the target temperature for a predetermined period of time (e.g., one hour); and
3. reducing the temperature of the chamber before the start of the coating process.

In some embodiments, the target temperature is 350° C. In some embodiments, the target temperature is maintained for 15-45 minutes. In some embodiments, the chamber temperature is reduced after the sterilization process.

### Process Using Steam (Wet Heat)

The process can include:
1. introduction of steam into the reactor chamber combined with suitable humidity control and turbulent airflow;
2. measurement and control of effective exposure to steam optimum for sterilization of the particles and the various surfaces of the reactor; and
3. removal of steam and any gaseous by-products from the reactor chamber before the start of the coating process.

As an example, the method according to the disclosure can include the following steps:
a) connecting a steam generator to the reactor chamber;
b) elevating and maintaining the steam concentration in the reactor chamber at a level sufficient to act as a disinfectant taking into account the humidity, size, temperature and airflow of the reactor chamber;
c) removing the steam and any gaseous by-products from the reactor chamber.

In some embodiments, steam can be introduced at a minimum temperature of 121.6° C. to reach a chamber pressure of 700 Torr. In some embodiments, the steam is kept in the chamber for 15-45 minutes. In some embodiments, steam is removed after the sterilization process.

In some embodiments, steam can be introduced at a temperature that is lower than 121.6° C. (e.g., 80° C.). In some embodiments, the steam is kept in the chamber for more than 5 hours (e.g., 6 hours). In some embodiments, steam is removed after the sterilization process.

### Process for Preparing Pharmaceutical Compositions Comprising Drugs Encapsulated By One Or More Layers Of Metal Oxide

After the particles are treated using either an ozone process (wet or dry) or an H₂O₂ process (wet or dry), they can be coated by, for example, one or more layers of a metal oxide or metalloid oxide. Provided are two exemplary methods for a pharmaceutical composition comprising a drug-containing core enclosed by one or more metal oxide materials. The first exemplary method includes the sequential steps of: (a) loading the particles comprising the drug into a reactor, (b) applying a vaporous or gaseous metal precursor to the substrate in the reactor, (c) performing one or more pump-purge cycles of the reactor using inert gas, (d) applying a vaporous or gaseous oxidant to the substrate in the reactor, and (e) performing one or more pump-purge cycles of the reactor using inert gas.

In some embodiments of the first exemplary method, the sequential steps (b)-(e) are optionally repeated one or more times to increase the total thickness of the one or more metal oxide materials that enclose the solid core of the coated particles. In some embodiments, the reactor pressure is allowed to stabilize following step (a), step (b), and/or step (d). In some embodiments, the reactor contents are agitated prior to and/or during step (b), step (c), and/or step (e). In some embodiments, a subset of vapor or gaseous content is pumped out prior to step (c) and/or step (e).

The second exemplary method includes (e.g., consists of) the sequential steps of (a) loading the particles comprising the drug into a reactor, (b) reducing the reactor pressure to less than 1 Torr, (c) agitating the reactor contents until the reactor contents have a desired moisture content, (d) pressurizing the reactor to at least 10 Torr by adding a vaporous or gaseous metal precursor, (e) allowing the reactor pressure to stabilize, (f) agitating the reactor contents, (g) pumping out a subset of vapor or gaseous content and determining when to stop pumping based on analysis of content in reactor including metal precursor and byproduct of metal precursor reacting with exposed hydroxyl residues on substrate or on particle surface, (h) performing a sequence of pump-purge cycles of the reactor using insert gas, (i) pressuring the reactor to at least 10 Torr by adding a vaporous or gaseous oxidant, (j) allowing the reactor pressure to stabilize, (k) agitating the reactor contents, (l) pumping out a subset of vapor or gaseous content and determining when to stop pumping based on analysis of content in reactor including metal precursor, byproduct of metal precursor reacting with exposed hydroxyl residues on substrate or on particle surface, and unreacted oxidant, and (m) performing a sequence of pump-purge cycles of the reactor using insert gas.

In some embodiments of the second exemplary method, the sequential steps (b)-(m) are optionally repeated one or more times to increase the total thickness of the one or more metal oxide materials that enclose the solid core of the coated particles.

### Pharmaceutically Acceptable Excipients, Diluents, and Carriers

Pharmaceutically acceptable excipients include, but are not limited to:
(1) surfactants and polymers including: polyethylene glycol (PEG), polyvinylpyrrolidone (PVP), sodium lauryl sulfate, polyvinylalcohol, crospovidone, polyvinylpyrrolidone-polyvinylacrylate copolymer, cellulose derivatives, hydroxypropylmethyl cellulose, hydroxypropyl cellulose, carboxymethylethyl cellulose, hydroxypropyllmethyl cellulose phthalate, polyacrylates and polymethacrylates, urea, sugars, polyols, carbomer and their polymers, emulsifiers, sugar gum, starch, organic acids and their salts, vinyl pyrrolidone and vinyl acetate;
(2) binding agents such as cellulose, cross-linked polyvinylpyrrolidone, microcrystalline cellulose;
(3) filling agents such as lactose monohydrate, lactose anhydrous, microcrystalline cellulose and various starches;
(4) lubricating agents such as agents that act on the flowability of a powder to be compressed, including colloidal silicon dioxide, talc, stearic acid, magnesium stearate, calcium stearate, silica gel;
(5) sweeteners such as any natural or artificial sweetener including sucrose, xylitol, sodium saccharin, cyclamate, aspartame, and acesulfame K;
(6) flavoring agents;
(7) preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quaternary compounds such as benzalkonium chloride;
(8) buffers;
(9) Diluents such as pharmaceutically acceptable inert fillers, such as microcrystalline cellulose, lactose, dibasic calcium phosphate, saccharides, and/or mixtures of any of the foregoing;
(10) wetting agents such as corn starch, potato starch, maize starch, and modified starches, and mixtures thereof;
(11) disintegrants; such as croscarmellose sodium, crospovidone, sodium starch glycolate; and
(12) effervescent agents such as effervescent couples such as an organic acid (e.g., citric, tartaric, malic, fumaric, adipic, succinic, and alginic acids and anhydrides and acid salts), or a carbonate (e.g., sodium carbonate, potassium carbonate, magnesium carbonate, sodium glycine carbonate, L-lysine carbonate, and arginine carbonate) or bicarbonate (e.g. sodium bicarbonate or potassium bicarbonate)

### EXAMPLES

### Example 1: Ozone Process

### 1) Dry Ozone Process

Powdered lactose having a bioburden of about 1800 CFU/ml was used to demonstrate the ability of the dry ozone process to reduce the bioburden of fine particles.

Briefly, 50 grams of lactose powder was loaded into the reactor chamber. The chamber was evacuated to reach a pressure of 0.008 Torr and then 0.01 Torr. Then the chamber was filled with ozone at 25 sccm to 2 ppm, 10 ppm or 20 ppm. The ozone was kept in the chamber for 5 hrs. The reaction was held at 35° C. and the reactor chamber was rotated at 10 rpm. At the end of the 5 hour incubation time, the chamber was evacuated to reach a pressure of 0.01 Torr and purged with nitrogen. The lactose was then tested using US Pharmacopeia 61 testing. Briefly, the samples were incubated in petri-dishes and counted for colony forming units (cfu). The results of this testing are presented in Table 1.

### 2) Wet Ozone Process

Powdered lactose having a bioburden of about 1800 CFU/ml was used to demonstrate the ability of the wet ozone process to reduce the bioburden of fine particles.

Briefly, 50 grams of lactose powder was loaded into the reactor chamber. The chamber was evacuated to reach a pressure of 0.008 Torr and then 0.01 Torr. Then the chamber was filled with ozone at 25 sccm to 2 ppm, 10 ppm or 20 ppm. Water vapor was also added at an ozone/water ratio of 1:10. The ozone was kept in the chamber for 5 hrs. The reaction was held at 35° C. and the reactor chamber was rotated at 10 rpm. At the end of the 5 hour incubation time, the chamber was evacuated to reach a pressure of 0.01 Torr and purged with nitrogen. The lactose was then test using US Pharmacopeia 61 testing. The results of this testing are presented in Table 1.

TABLE 1
Results of Dry Ozone Process and Wet Oxone Process Testing

| | | |
|---|---|---|
| | Ozone | |
| Process | Concentration | Bioburden |

| | | | | |
|---|---|---|---|---|
| Dry ozone | 0 | ppm | 1800 | CFU/ml |
| | 2 | ppm | 500 | CFU/ml |
| | 10 | ppm | 50 | CFU/ml |
| | 20 | ppm | <1 | CFU/ml |
| Wet Ozone | 0 | ppm | 1800 | CFU/ml |
| | 2 | ppm | 500 | CFU/ml |
| | 10 | ppm | 50 | CFU/ml |
| | 20 | ppm | <1 | CFU/ml |

1. A method of applying a coating to particles comprising an active pharmaceutical ingredient (API), the method comprising:
(a) loading the particles into a chamber;
(b) applying a vaporous or gaseous metal or metalloid precursor to the particles in the reactor by pulsing the vaporous or gaseous aluminum precursor into the reactor;
(c) performing one or more pump-purge cycles using an inert gas;
(d) applying a vaporous or gaseous oxidant to the particles in the reactor by pulsing the oxidant into the reactor;
(e) performing one or more pump-purge cycles using an inert gas;
(f) repeating steps (a)-(e) at least once to provide a metal oxide or metalloid oxide coating layer on the particles; and a sterilization step before step (a), between step (a) and step (b), and/or after step (f).

2. The method of claim 1, wherein the sterilization step comprises isolating the chamber from the ambient environment.

3. The method of claim 1, wherein the method comprises a sterilization step between step (a) and step (b).

4. The method of claim 1, wherein the sterilization step comprises: injecting ozone into the chamber to reach a predetermined ozone concentration and holding for a predetermined period of time.

5. The method of claim 4, wherein the sterilization step further comprises removing the ozone after the expiry of the predetermined period of time.

6. The method of claim 5, wherein the removing further includes injecting an inert gas into the chamber.

7. The method of claim 6, wherein the inert gas is nitrogen, argon, or helium.

8. The method of claim 4, wherein the predetermined ozone concentration is 9-19 ppm, and the method further comprises injecting water vapor into the chamber to reach a predetermined steam pressure and holding for a predetermined period of time.

9. The method of claim 4, wherein the predetermined ozone concentration is 30-120 ppm.

10. The method of claim 4, wherein the predetermined period of time is less than 1 hour.

11. The method of claim 4, wherein the predetermined period of time is 3-5 hours.

12. The method of claim 4, wherein the predetermined period of time is 5-10 minutes, and the method further comprises injecting water vapor into the chamber to reach a predetermined steam pressure and holding for a predetermined period of time.

13. The method of claim 1, wherein the sterilization step comprises: injecting H₂O₂ vapor into the chamber to reach a predetermined H₂O₂ concentration and holding for a predetermined period of time.

14.-16. (canceled)

17. The method of claim 13, wherein the predetermined H₂O₂ concentration is 100-500 ppm.

18. The method of claim 13, wherein the predetermined H₂O₂ concentration is 400-6000 ppm, and the method further comprises injecting water vapor into the chamber to reach a predetermined steam pressure and holding for a predetermined period of time.

19.-21. (canceled)

22. The method of claim 1, wherein the sterilization step comprises increasing the temperature of the chamber to reach a target temperature and maintaining the target temperature for a predetermined period of time.

23. The method of claim 22, wherein the target temperature is 350° C.

24.-26. (canceled)

27. The method of claim 1, wherein the sterilization step comprises: injecting water vapor into the chamber to reach a predetermined steam pressure and holding for a predetermined period of time.

28. (canceled)

29. The method of claim 27, wherein the predetermined steam pressure is 700 Torr.

30.-32. (canceled)

33. The method of claim 1, wherein the bio-burden of the particles is reduced by more than 90%.

34.-39. (canceled)
